(19) United States
(12) Patent Application Publication
(10) Pub. No.: US 2008/0317725 A1
(43) Pub. Date: Dec. 25, 2008

(54) COMPOSITIONS AND METHODS OF TREATING CHRONIC KIDNEY DISEASE

(76) Inventor: Seth J. Baum, Boca Raton, FL (US)

Correspondence Address: MORRISS OBRYANT COMPAGNI, P.C., 734 EAST 200 SOUTH, SALT LAKE CITY, UT, 84102, US

(21) Appl. No.: 12/143,022

(22) Filed: Jun. 20, 2008

Related U.S. Application Data
(60) Provisional application No. 60/936,544, filed on Jun. 20, 2007.

(51) Int. Cl. A61K 9/48; A61K 38/16; A61K 38/43; A61K 33/34; A61P 21/00; A61P 13/12; A61K 31/375; A61K 31/714

(52) U.S. Cl. 424/94.1; 514/12; 514/52; 424/463; 514/474; 424/638

(57) ABSTRACT

The invention relates to nutritional compositions and methods of using these compositions for the treatment of renal disease. More particularly, the invention discloses compositions of vitamins, minerals, amino acids, and/or proteins in amounts that can be used to supplement the nutritional deficiencies observed in patients afflicted with renal disease, renal insufficiency, or end-stage renal disease. The compositions of the invention can also be used as nutritional supplements for patients undergoing dialysis therapy or for patients on a restricted diet. In addition, the compositions can be used in combination or alone as a method of treating or managing a subject in the various stages of chronic renal disease, or throughout disease progression.

### CROSS-REFERENCE TO RELATED APPLICATIONS

This application claims the benefit of U.S. Provisional Application No. 60/936,544, filed Jun. 20, 2007, which is incorporated by reference.

### TECHNICAL FIELD

This invention relates to the treatment or management of subjects with kidney disease, more particularly, to compositions and methods of treating or managing patients suffering from kidney disease.

### BACKGROUND

The kidneys perform a variety of physiological functions, including controlling fluid and electrolyte homeostasis, excretion of nitrogenous wastes, secretion of erythropoietin, and production of 1,25-dihydroxy vitamin D3. Therefore, loss of renal function can affect multiple organ systems. The loss of renal function, or renal disease, is generally classified as either acute renal failure or chronic renal failure.

Acute renal failure is generally characterized by a rapid and sometimes reversible reduction or cessation in renal function. Chronic renal failure, which is a progressive irreversible loss of renal function, may be caused by immunological disorders, such as glomerulonephritis, and metabolic disorders such as diabetes mellitus (responsible for over 50% of end stage renal disease cases) and hypertension. Progressive deterioration of kidney function in chronic renal failure can lead to end-stage renal failure, which results in toxins accumulating in the body that must be removed by dialysis.

Hemodialysis is a process where blood is removed from the body and pumped into a dialyzer (artificial kidney), which filters metabolic waste products from the blood and then returns the purified blood to the subject. Peritoneal dialysis uses the peritoneum as a filter. In Peritoneal Dialysis, a dialysate is infused through a catheter inserted through the abdominal wall into the peritoneal space within the abdomen, left in the abdomen for a sufficient time to allow a concentration gradient dependent exchange of the waste products from the bloodstream of the abdomen, and then the dialysate is drained out, discarded, and replaced with fresh dialysate. Typically, hemodialysis treatment is performed about three times a week, whereas peritoneal dialysis is nearly a continuous process.

End stage renal disease patients on chronic Hemodialysis suffer from chronic inflammation and malnutrition. Their life expectancy is reduced 20 to 25 years and they have a 10-fold higher risk of cardiovascular death. The annual mortality of chronic dialysis patients is 20%. Dialysis typically results in the subject having a poor appetite, altered taste sensation, and an aversion to high protein foods such as meat, with protein being lost during dialysis. Three grams of protein and 4 to 8 grams of amino acids are lost during a typical dialysis session and catabolism persists for 2 hours following dialysis. Therefore, subjects undergoing dialysis need a diet relatively high in protein (especially on the days of treatment), while both sodium and potassium intake is restricted. In hemodialysis, daily consumption of sodium and potassium is even more restricted. Foods high in phosphorus and calcium also may have to be limited and fluid intake is frequently severly restricted. Therefore, there is a need for a low fluid, low phosphorus, low sodium, low calcium protein source, such as a specifically-designed protein bar.

Multivitamin supplements are typically needed to replace the nutrients lost through hemodialysis or peritoneal dialysis, but uncontrolled or improper use of multivitamins and multiminerals can lead to additional problems. For example, in subjects undergoing dialysis a vitamin A intake of about 15,000 IU was been found to produce vitamin A toxicity and correlate with hypercalcaemia.¹

The fact that dialysis patients have difficulty in obtaining proper dietary amounts of essential vitamins and minerals has resulted in the formulation of vitamin and nutrient supplements for renal patients. Products currently on the market include DIATX (Pamlab, LLC), RENAX Caplets (Everett Laboratories, Inc.) and NEPHROCPS (Fleming & Company). These vitamin formulations contain water soluble vitamins such as folic acid, biotin, niacin, pantothenic acid (vitamin B5), thiamine (vitamin B1), riboflavin (vitamin B₂), pyridoxine (vitamin B6), vitamin B₁₂ (cyanocobalamin), vitamin C (ascorbic acid), as well as the minerals selenium and zinc in various amounts. However, one formulation does not fit all situations.

Chronic Kidney Disease (CKD) affects one in nine adults, or 20 million Americans. Based upon the kidney's ability to filter the blood (known as the Glomerular Filtration Rate, or GFR) CKD is divided into five progressive stages. The GFR is calculated using a mathematical formula that factors in the subject's age, race, gender and their serum creatinine levels.

Stages one and two are usually so mild that they may go undetected and usually do not require, but may benefit from, specific nutritional adjustments, although stage two subjects may be advised to reduce their salt intake. Stage four (severe CKD), and often stage 3 (moderate CKD) may require changes in diet and medications. These patients typically have to modify their intake of protein, phosphorus, calcium and other minerals. As Diabetes and Hypertension represent the leading causes of CKD, patients typically should also limit simple sugars, maintain ideal body weight, and keep sodium intake at reasonably low levels. End stage CKD, or stage five, requires dialysis or some form of renal replacement therapy to prolong life. Patients on dialysis have nutritional needs that differ from their less advanced counterparts. They need more protein and replacement of vitamins and minerals that are lost to dialysis.

While there is currently no cure for CKD, it is possible to delay progression of the disease. In many cases, lifestyle changes, such as dietary intake, can help maintain kidney function. However, until this point there has been no organized nutritional approach for all stages of CKD from a vitamin/mineral supplement standpoint.

The present invention provides a number of compositions and methods of treating patients in all stages of CKD. The invention has beneficial aspects for patients diagnosed with hyperhomcysteinemia, which is common in dialysis patients.

### SUMMARY OF THE INVENTION

The invention provides nutritional compositions and methods of using these compositions for the treatment of a subject with renal disease. More particularly, the invention discloses compositions of vitamins, minerals, amino acids, and/or proteins in an amount that can be used to supplement the nutritional deficiencies observed in patients afflicted with renal disease, renal insufficiency, or end-stage renal disease. The compositions of the invention can also be used as nutritional supplements for patients undergoing dialysis therapy or for patients on a restricted diet. In addition, the compositions can be used in combination or alone as a method of treating or managing a subject in the various stages of chronic renal disease, or throughout disease progression. Hence, the invention provides a suite of compostions that may be used in the treatment or management of renal disease.

The compositions of the invention comprise numerous vitamins, minerals, amino acids and protein in various combinations that will improve the nutritional state of a subject. The vitamins included in the compositions of the present invention may include vitamin C, vitamin E, vitamin B₁, vitamin B₂, vitamin B₃, vitamin B₅, vitamin B₆, vitamin B₁₂, biotin, vitamin B₉, vitamin D and vitamin E. The minerals included in the compositions of the invention may include one or more of zinc, copper, chromium, selenium, manganese, and boron. The amino acids and/or proteins included in the compositions of the invention may include N-Acetyl Cysteine, Glutathione, isoleucine, leucine, lysine, methionine, phenylalanine, histidine, threonine, tryptophan, and/or valine.

In an exemplary embodiment, the compositions may comprises one or more of vitamin A in the form of beta carotene; vitamin C in the form of ascorbic acid; vitamin D in the form of cholecalciferol; vitamin E in the form of d-alpha tocopheryl and/or mixed tocopherols; vitamin B₁ in the form of thiamine mononitrate; vitamin B₂ in the form of riboflavin; vitamin B₃ in the form of niacinamide and/or niacin; vitamin B₅ in the form of pantothenic acid (d-calcium pantothenate); vitamin B₆ in the form of pyridoxine hydrochloride; vitamin B₁₂ in the form of cyanocobalamin; biotin; vitamin B₉ in the form of folic acid, folacin, metafolin, folate and/or one or more natural isomers of folate including (6S)-tetrahydrofolic acid or a polyglutamyl derivative thereof, 5-methyl-(6S)-tetrahydrofolic acid or a polyglutamyl derivative thereof, 5-formyl-(6S)-tetrahydrofolic acid or a polyglutamyl derivative thereof 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof; vitamin K in the form of phytonadione (K1 and K2 may be used); calcium in the form of calcium carbonate and/or calcium citrate; iodine in the form of potassium iodine; molybdenum in the form of molybdenum amino acid chelate; magnesium in the form of magnesium oxide and/or magnesium amino acid chelate; copper in the form of copper amino acid chelate; zinc in the form of zinc amino acid chelate and/or zinc oxide; chromium in the form of chromium amino acid chelate; selenium in the form of L-selenomethionine and/or selenium amino acid chelate; manganese in the form of manganese amino acid chelate; potassium; boron in the form of boron amino acid chelate and lycopene.

In another exemplary embodiment of the present invention, the compositions may be substantially free of one or more added vitamins and minerals not described in the preceding paragraph. For example, the compositions of the present invention may be substantially free of added lutein; substantially free of added zeaxanthin; substantially free of added vitamin B₄; substantially free of added vitamin B₁₀; substantially free of added calcium; substantially free of added iron; substantially free of added odorless garlic; substantially free of added coenzyme Q-10; substantially free of added 1-carnitine; substantially free of added quercetin; substantially free of added starch; substantially free of added yeast; substantially free of added sugar, substantially free of added alpha lipoic acid and/or combinations thereof.

In another exemplary embodiment, the composition comprises CoQ10; L-carnitine; lipoic acid; vitamin E; and resveratrol, which may be administered to a subject suffering from weakness, fatique and/or cramping associated with kidney disease. In another exemplary embodiment, the composition comprises COQ10, L-carnitine and lipoic acid that may be administered to a subject suffering from weakness, fatique and/or cramping associated with kidney disease and the common use of Statin medications.

In another exemplary embodiment, the composition comprises N-acetyl cysteine (NAC) and vitamin C, which may be particularly advantages when administered to a subject scheduled to receive contrast media.

The invention also includes methods for supplementing nutritional deficiencies in patients that have nutritional deficiencies due to kidney disease, end-stage renal disease, renal insufficiency, dialysis therapy, dietary restrictions or other disease states that result in increased oxidative stress, elevated cholesterol levels, and/or elevated homocysteine levels.

The invention provides a treatment system for the treatment of renal disease in a subject where the system comprises at least two, at least three, or at least four compositions described herein. For example, the system may include compositions selected from: the vitamin formulations described herein; compositions comprising CoQ10, L-carnitine, lipoic acid, vitamin E, and resveratrol; compositions comprising N-acetyl cysteine (NAC); compositions comprising phytosterols; and a high protein food bar described herein. Vitamin formulations of the invention comprise vitamin C, vitamin D₃, vitamin E, vitamin B₁, vitamin B₂, vitamin B₃, vitamin B₆, vitamin B₁₂, vitamin B₁, folic acid, biotin, pantothenic acid, zinc, selenium and chromium. Optionally, the a vitamin formulation may also comprise NAC. Compositions of the invention include vitamin formulations having about 200% of the Recommended Daily Value (RDV) of vitamin B₁ and B₂, greater than about 500% of the RDV of vitamin B6, at least about 16,000% of the RDV of vitamin B12, and at least about 750% of the RDV of folic acid (vitamin B9).

The invention also relates to a high protein food bar, that optionally may be a medical food, wherein the high protein food bar has at least about 20 grams of protein per 60 gram high protein food bar, 5-10% by weight water, 10-30% by weight glycerine, 10-30% by weight manitol syrup, less than about 2% lecithin, 10-30% by weight whey protein, 10-30% by weight calcium caseinate, 5-10% by weight soy protein and less than about 2% wheat germ, less than about 2 grams of sugar per 60 gram high protein food bar and less than about 230 mg of sodium and less than about 240 mg of potasium. Optionally, the high protein food bar may contain a low level of phosphorous, for example, less than about 20% of the recommended daily value (based on a 2,000 calorie diet) of phosphorous, less than about 18%, or less than about 15%.

In another exemplary embodiment, the invention provides a high protein food bar that may be used to supplement protein intake, particularly in patients undergoing Hemodialysis or peritoneal dialysis. The high protein food bar may have a low level of sugar (e.g, less than about 5 g, less than about 4 g, less than about 3 g, less than about 2 g, or less than about 1 g of sugar per bar). In an exemplary embodiment, the PDCAAS score of the high protein food bar will be approximately 1, reflecting an optimal protein source. Optionally, the high protein food bar will not contain an artificial sweetener and/or added vitamins.

In another exemplary embodiment, the high protein food bar is made using a polyol, has a low sugar content (e.g., about 1 gram per bar), does not contain any non-nutritive seateners, without vitamin fortification or addition, and has a PDCAAS value of about 1.

The invention also provides a method of providing protein to a subject undergoing dialysis treatment or about to undergo dialysis treatment, wherein a high protein food bar having a low sugar content (e.g., about 1 gram per bar), no non-nutritive seateners, no added vitamin fortification, and/or a PDCAAS value of about 1 is administered to the subject. In an exemplary embodiment the high protein food bar is consumed by the subject during dialysis treatment to treat or prevent skeletal muscle wasting.

### DETAILED DESCRIPTION OF THE INVENTION

As used herein and in the appended claims, “about” means reasonably close to, or approximately, a little more or less than the stated number or amount.

As used herein and in the appended claims, a “Subject” refers to a mammal, including a human, cat, dog, or horse, and includes a “patient.”

As used herein and in the appended claims, the singular forms “a”, “an”, and “the” include plural reference unless the context clearly dictates otherwise.

“Treating” or “treatment” does not require a complete cure. It means that a symptom of the underlying disease is at least reduced, and/or that one or more of the underlying cellular, physiological, or biochemical causes or mechanisms causing the symptom are reduced and/or eliminated. It is understood that reduced, as used in this context, means relative to the state of the disease, including the molecular state of the disease, not just the physiological state of the disease

As used herein, “comprising,” “including,” “containing,” “characterized by,” and grammatical equivalents thereof are inclusive or open-ended terms that do not exclude additional, unrecited elements or method steps, but also includes the more restrictive terms “consisting of” and “consisting essentially of.”

In an exemplary embodiment, the composition may comprise vitamin A in the form of beta carotene at an amount of about 100% of the FDA's Recommended Daily Value or Recommended Daily Intake (RDV, RDI respectively, herein referred to as RDV based on a 2,000 calorie intake); vitamin C in the form of ascorbic acid at an amount of about 100% of the RDV; vitamin D₃ in the form of cholecalciferol at an amount of about 100% of the RDV; vitamin E in the form of d-alpha tocopheryl and/or mixed tocopherols at an amount of about 100% of the RDV; vitamin B₁ in the form of thiamine mononitrate at an amount of about 100% of the RDV; vitamin B₂ in the form of riboflavin at an amount of about 100% of the RDV; vitamin 33 in the form of niacinamide and/or niacin at an amount of about 100% of the RDV; vitamin B₅ in the form of pantothenic acid (d-calcium pantothenate) at an amount of about 100% of the RDV; vitamin B₆ in the form of pyridoxine hydrochloride at an amount of about 100% of the RDV; vitamin B₁₂ in the form of cyanocobalamin at an amount of about 100% of the RDV; biotin at an amount of about 100% of the RDV; vitamin B₉ in the form of folic acid at an amount of about 100% of the RDV; vitamin K in the form of phytonadione at an amount of about 100% of the RDV; calcium in the form of calcium carbonate and/or calcium citrate at an amount of about 100% of the RDV; iodine in the form of potassium iodine at an amount of about 100% of the RDV; molybdenum in the form of molybdenum amino acid chelate at an amount of about 100% of the RDV; magnesium in the form of magnesium oxide and/or magnesium amino acid chelate at an amount of about 100% of the RDV; copper in the form of copper amino acid chelate at an amount of about 100% of the RDV; zinc in the form of zinc amino acid chelate and/or zinc oxide at an amount of about 100% of the RDV; chromium in the form of chromium amino acid chelate at an amount of about 100% of the RDV; selenium in the form of L-selenomethionine and/or selenium amino acid chelate at an amount of about 100% of the RDV; manganese in the form of manganese amino acid chelate at an amount of about 100% of the RDV; potassium at an amount of about 51 mg; chloride at an amount of about 46 mg; boron in the form of boron amino acid chelate at an amount of about 3 mg; and lycopene at an amount of about 6 mg.

In another exemplary embodiment, the composition may comprise vitamin A in the form of beta carotene at an amount of about 100% of the RDV; vitamin C in the form of ascorbic acid at an amount of about 100% of the RDV; vitamin D₃ in the form of cholecalciferol at an amount of about 100% of the RDV; vitamin E in the form of d-alpha tocopheryl and/or mixed tocopherols at an amount of about 100% of the RDV; vitamin B₁ in the form of thiamine mononitrate at an amount of about 100% or the RDV; vitamin B₂ in the form of riboflavin at an amount of about 100% of the RDV; vitamin B₃ in the form of niacinamide and/or niacin at an amount of about 100% of the RDV; vitamin B₅ in the form of pantothenic acid (d-calcium pantothenate) at an amount of about 100% of the RDV; vitamin B₆ in the form of pyridoxine hydrochloride at an amount of about 100% of the RDV; vitamin B₁₂ in the form of cyanocobalamin at an amount of about 100% of the RDV; biotin at an amount of about 100% of the RDV; vitamin B₉ in the form of folic acid at an amount of about 100% of the RDViodine in the form of potassium iodine at an amount of about 100% of the RDV; molybdenum in the form of molybdenum amino acid chelate at an amount of about 100% of the RDV; magnesium in the form of magnesium oxide and/or magnesium amino acid chelate at an amount of about 100% of the RDV; copper in the form of copper amino acid chelate at an amount of about 100% of the RDV; zinc in the form of zinc amino acid chelate and/or zinc oxide at an amount of about 100% of the RDV; chromium in the form of chromium amino acid chelate at an amount of about 100% of the RDV; selenium in the form of L-selenomethionine and/or selenium amino acid chelate at an amount of about 100% of the RDV; manganese in the form of manganese amino acid chelate at an amount of about 100% of the RDV; potassium at an amount of about 99 mg; chloride at an amount of about 95 mg; boron in the form of boron amino acid chelate at an amount of about 3 mg, lycopene at an amount of about 6 mg; is substantially free of vitamin K; and calcium in the form of calcium carbonate and/or calcium citrate at an amount of about 50% of the RDV.

In another exemplary embodiment, the composition may comprise a formulation that may be used for the treatment of endstage renal failure, wherein the composition comprises vitamin C in the form of ascorbic acid at an amount of about 200% of the RDV; vitamin D₃ in the form of cholecalciferol at an amount of about 100% of the RDV; vitamin E in the form of d-alpha tocopheryl and/or mixed tocopherols at an amount of about 100% of the RDV; vitamin B₁ in the form of thiamine mononitrate at an amount of about 200% of the RDV; vitamin B₂ in the form of riboflavin at an amount of about 200% of the RDV; vitamin B₃ in the form of niacinamide and/or niacin at an amount of about 100% of the RDV; vitamin B₅ in the form of pantothenic acid (d-calcium pantothenate) at an amount of about 200% of the RDV; vitamin B₆ in the form of pyridoxine hydrochloride at an amount of about 1,250% of the RDV; vitamin B₁₂ in the form of cyanocobalamin at an amount of about 33,333% of the RDV; biotin at an amount of about 100% of the RDV; vitamin B₉ in the form of folic acid at an amount of about 1,250% of the RDV; zinc in the form of zinc amino acid chelate and/or zinc oxide at an amount of about 100% of the RDV; chromium in the form of chromium amino acid chelate at an amount of about 50% of the RDV; selenium in the form of L-selenomethionine and/or selenium amino acid chelate at an amount of about 50% of the RDV; and N-Acetyl cysteine in an amount of about 400 to about 600 mg, or about 500 mg.

In another exemplary embodiment, the composition may comprise a formulation that may be used for the treatment of stage 3 or stage 4 kidney disease, wherein the composition may comprise vitamin C in the form of ascorbic acid at an amount of about 200% of the RDV; vitamin D₃ in the form of cholecalciferol at an amount of about 100% of the RDV; vitamin E in the form of d-alpha tocopheryl and/or mixed tocopherols at an amount of about 100% of the RDV; vitamin B₁ in the form of thiamine mononitrate at an amount of about 200% of the RDV; vitamin B₂ in the form of riboflavin at an amount of about 200% of the RDV; vitamin B₃ in the form of niacinamide and/or niacin at an amount of about 50% of the RDV; vitamin B₅ in the form of pantothenic acid (d-calcium pantothenate) at an amount of about 100% of the RDV; vitamin B₆ in the form of pyridoxine hydrochloride at an amount of about 500% of the RDV; vitamin B₁₂ in the form of cyanocobalamin at an amount of about 16,667% of the RDV; biotin at an amount of about 100% of the RDV; vitamin B₉ in the form of folio acid at an amount of about 750% of the RDV; zinc in the form of zinc amino acid chelate and/or zinc oxide at an amount of about 100% of the RDV; chromium in the form of chromium amino acid chelate at an amount of about 25% of the RDV; selenium in the form of L-selenomethionine and/or selenium amino acid chelate at an amount of about 50% of the RDV; copper in the form of copper amino acid chelate at an amount of about 20-40% (or 25%) of the RDV; chromium in the form of chromium amino acid chelate at an amount of about 25% of the RDV; and N-Acetyl cysteine in an amount of about 400 mg to about 600 mg, or about 500 mg.

Hyperhomcysteinemia is characterized by high serum homocysteine levels that can be lowered with a combination of folic acid, vitamin B₆ and vitamin B₁₂ which function by converting homocysteine to methionine. Therefore, the compositions of the invention that have higher levels of one or more of these compounds may be beneficially used to treat or manage subjects suffering from elevated homocysteine levels. For example, compositions of the invention may comprise about 1,250% of the RDV of vitamin B₆, about 750% of the RDV of folate, and about 16,667% of the RDV of vitamin B₁₂. In an other exemplary embodiment, the composition may comprise about 200% of the RDV of vitamin B₁, 200% of the RDV of vitamin B₁, at least about 500% of the RDV of vitamin B₆ or at least 1,000% of the KDV of vitamin B₆, at least about 16,000% of the RDV of vitamin B₁₂ or at least about 33,000% of the RDV of vitamin B₁₂, at least 750% of the RDV of folic acid (vitamin B₉) or at least about 1,250% of the RDV of folic acid, and a combination thereof. Optionally, the vitamin compositions may be fortified with NAC, for example between about 400 and about 600 mg, or about 500 mg. In another exemplary embodiment, the invention provides a composition comprising about 25 mg (about 1250% RDV) of vitamin B₆, about 3 mg (about 750% RDV) of folate, and about 1 mg (about 16,667% RDV) of vitamin B₁₂, which may be administered once a day.

The embodiments of the invention described herein may be administered to a patient one or more times daily. These embodiments may also be administered orally and may comprise pharmaceutically acceptable carriers, excipients and/or diluents. It is contemplated that these formulations can be used to treat nutritional deficiencies in patients requiring such treatment due to kidney disease, end-stage renal disease, renal insufficiency, dialysis therapy, dietary restrictions, elevated homocysteine levels, or other disease states.

In an exemplary embodiment, the formula describe at paragraph [0032] may be administered daily to a patient on dialysis, particulary subjects with end stage kidney disease. In another exemplary embodiment, the formula described at paragraph [0033] may be administered daily to a patient with stage 3 or stage 4 CKD. In yet another exemplary embodiment, the formula describe at paragraph [0033] is administered to a patient with stage 3 or stage 4 CKD and the patient is switched to the formula described at paragraph [0032] when the patient begins dialysis.

In another exemplary embodiment, the composition comprises CoQ₁₀ in a range of about 4 to about 10 percent by weight, about 4.5 to about 9.5% (wt:wt), about 5 to about 9% (wt:wt); about 5.5 to about 8.5% (wt:wt); about 6 to about 8% (wt:wt); about 6.5 to about 7.5% (wt:wt); or about 7% (wt:wt); L-carnitine in a range of about 50 to about 90% (wt:wt), about 55 to about 85% (wt:wt), about 60 to about 80% (wt:wt); about 65 to about 75% (wt:wt); or about 70% (wt:wt); lipoic acid in a range of 4 to about 10% (wt:wt), about 4.5 to about 9.5% (wt:wt), about 5 to about 9% (wt:wt); about 5.5 to about 8.5% (wt:wt); about 6 to about 8% (wt:wt); about 6.5 to about 7.5% (wt:wt); or about 7% (wt:wt); vitamin E in a range of about 0.5 to about 3.5% (wt:wt), about 1 to about 3% (wt:wt); about 1.5 to about 2.5% (wt:wt); or about 2% (wt:wt); and resveratrol in a range of about 4 to about 24% (wt:wt), about 6 to about 22% (wt:wt), about 9 to about 19% (wt:wt); about 11 to about 17% (wt:wt); about 13 to about 15% (wt:wt); or about 14% (wt:wt).

In another exemplary embodiment, the composition comprises CoQ₁₀ at about 50 mg; L-carnitine at about 500 mg; lipoic acid at about 50 mg; vitamin E at about 30 IU (or about 100% RDV); and resveratrol at about 100 mg. In yet another exemplary embodiment, the composition comprises CoQ₁₀ at about 50 mg; L-carnitine at about 400 mg; lipoic acid at about 50 mg; vitamin E at about 30 IU (or about 100% RDV); and resveratrol at about 50 mg. These compositions may be administered once a day.

In another exemplary embodiment, the composition may be used to treat subjects, particularly dialysis patients, who develop weakness, fatigue and muscular cramping. Compositions containing L-carnitine may beneficial treat subjects with these syptoms or to improve non-response to erythropoietin and treat intradialytic hypotension.

Contrast induced nephropathy typically occurs within 3 days of the administration of contrast medium and may be measured by an increase in serum Creatinine of more than 0.5 mg/dl or 25% above baseline. Contrast induced nephropathy is the third most common cause of acute renal failure in patients admitted to the hospital, with patients having preexisting renal impairment or diabetes mellitus having a 20% to 80% greater risk.² Therefore, this population is particularly susceptible to developing contrast induced nephropathy. Exemplary embodiments described herein may be particularly advantages when administered to a subject scheduled to receive contrast media.

In another exemplary embodiment, the composition comprises N-acetyl cysteine (NAC) at about 1,200 mg and vitamin C at about 250 mg to about 750 mg, for example at about 500 mg or about 600 mg. In yet another exemplary embodiment, the composition comprises N-acetyl cysteine (NAC) at about 600 mg. This exemplary embodiment may be particularly advantages when administered to a subject scheduled to receive contrast media. Compositions containing NAC may be contained in an enteric coated gel capsule.

In another exemplary embodiment the composition comprises N-acetyl cysteine, for example, at about 1,200 mg, and vitamin C, for example, at about 500 or 600 mg, in a specific blister pack arrangement to facilitate and increase user compliance. For example, a blister package having three rows and three columns appropriately marked for easy identification and use. For example, row 1 may be conspicuously marked with “AM,” “Morning,” “Breakfast” or other such language indicating the early portion of the user's day; row 2 may be conspicuously marked with “Lunch,” “Mid-day” or other such language indicating the middle portion of the user's day; and row 3 may be conspicuously marked with “Dinner,” “PM,” “Evening” or other such language indicating the last portion of the user's day, In this example, two enteric coated 600 mg NAC capsules are placed in each column of row 1 and row 3 and one 500 mg vitamin C capsule or tablet is placed in each column of row 2. The columns are marked in such a way that the user can clearly identify each column as being associated with a single day. Thus, the blister pack contains an AM dosage of NAC, a Lunch dosage of vitamin C, and a PM dosage of NAC laid out in an easy to use format (increase subject compliance) that is also easy to store and transport. A subject may take the first day's dosages at the appropriate times during the day without having to carry multiple containers or remember which pills or capsules, and what number of them, should be taken at specific times of the day. Further, it is desirable to administer the first day's dosage one day prior to undergoing a contrast study, the second day's dosage on the day of the constrast study and the third day's dosage the day following the contrast study. Therefore, one blister pack supplies the entire treatment course and can easily be carried and used by the subject. In another exemplary embodiment, the vitamin C is admixed with the AM and/or PM dose of NAC and the blister pack is reduced to two rows and three columns, however, given the decrease in absorption that vitamin C may cause, it may be more beneficial to separate the administration of vitamin C. Furthermore, NAC has been reported to cause nausea when administered in large doses, therefore, the invention contemplates the use of an enteric coated capsule or tablet for at least the NAC to reduce the undesirable side effects and/or increase intestinal absorption.

Contrast induced nephropathy is a common problem among CKD patients undergoing CT scans. The present invention provides a composition and packaging system that is easy to use, increasing compliance, and that can be provided to patients scheduled to undergo a CT scan by the imaging facility or by the physician requesting the scan. Contrast-Induced Nephropathy (CIN) may be marked by an absolute increase in serum creatinine of at least 0.5 mg/dL in patients with baseline serum creatinine levels less than or equal to about 2 mg/dL, a resultant increase in serum creatinine of at least 25% from baseline, and a decrease in GFR greater than 25%. Contrast-induced nephropathy occurs in approximately 15% of radio-contrast procedures. Mucomyst is a brand of acetylcysteine liquid formulation that is currently provided to some patients. However, consumption of a liquid formulation of NAC is unpleasant at best and Mucomyst is only available by perscription. The present invention provides a non-perscription formulation that avoids the unpleasant taste, thereby increasing compliance and providing a superior product for the prevention of CIN.

Vitamin D is a fat-soluble vitamin that is essential for maintaining normal calcium metabolism. Vitamin D3 (cholecalciferol) can be synthesized by humans in the skin upon exposure to ultraviolet-B (UVB) radiation from sunlight, or it can be obtained from the diet. Plants synthesize vitamin D2 (ergocalciferol), which also has vitamin D activity in humans, although this activity is about 1.7 times less than D3. When exposure to UVB radiation is insufficient for the synthesis of adequate amounts of vitamin D3 in the skin, adequate intake of vitamin D from the diet is essential for health.

Vitamin D itself is biologically inactive, and it must be metabolized to its biologically active forms. After it is consumed in the diet or synthesized in the skin, vitamin D enters the circulation and is transported to the liver. In the liver, vitamin D is hydroxylated to form 25-hydroxyvitamin D L25(OH)D], the major circulating form of vitamin D. Increased exposure to sunlight or increased intake of vitamin D increases serum levels of 25(OH)D, making the serum 25(OH)D concentration a useful indicator of vitamin D nutritional status. In the kidney and other tissues, the 25(OH)D3-1-hydroxylase enzyme catalyzes a second hydroxylation of 25(OH)D, resulting in the formation of 1,alpha,25-dihydroxyvitamin [1,25(OH)2D] (vitamin D₃), which is the most potent form of vitamin D.

In another exemplary embodiment, the composition comprises vitamin D at an amount of about 800 IU (or about 200% RDV), preferably the vitamin D is vitamin D₃ (cholecalciferol). In an exemplary embodiment, this composition is administered to subjects having low serum vitamin D levels and/or abnormal bone and mineral metabolism. For example, this composition may be administered to a subset of subjects using other compositions of the invention. In another exemplary embodiment, the composition comprises about 100 IU of vitamin D3 (as cholecalciferot), about 40 μg vitamin K1 (as phylloquinone), about 40 μg vitamin K2 (as menaquinone), and about 200 mg Calcium (as calcium citrate). Optionally the composition may also comprises hydroxypropyl methylcellulose and/or magnesium stearate.

The invention also includes methods for supplementing nutritional deficiencies in patients that have nutritional deficiencies due to kidney disease, end-stage renal disease, renal insufficiency, dialysis therapy, dietary restrictions or other disease states that result in increased oxidative stress, elevated cholesterol levels, and/or elevated homocysteine levels. In another exemplary embodiment, the invention provides a method of treating patients with CKD through all stages of the disease, for example, the composition described in paragraphs [0030] and [0031] is administered to healthy subjects or subjects having stage 1 or stage 2 CKD, as the subjects condition progresses to stage 3 or stage 4 CKD, the subject is switched to the composition describe at paragraph [0033], and about the time the subject begins dialysis the subject is switched to the composition described at paragraph [0032]. Subjects that experience weakness, fatique and/or muscular cramping may also be administered the composition described at paragraphs [0037] or [0038]. Subjects scheduled to receive contrast media may also be administered the composition described at paragraphs [0041] and/or [0042], which may be packaged as describe herein, and those subjects needing additional vitamin D may be treated using the composition described in paragraph [0046].

In another exemplary embodiment, a composition of the invention may comprise about 800 mg of Phytosterols (std to 90% total sterols) (as free sterols), about 300 mg SYTRINOL® (a blend of citrus polymethoxylated flavones and palm tocotrienols), about 20 mg BIOCOSANOL® (std to 90% Policosanol—sugar cane derived). Optionally the composition may also comprise microcrystalline cellulose, calcium silicate, croscarmellose sodium, stearic acid, magnesium stearate, hydroxypropyl methylcellulose, triacetin food grade, talc, chlorophyll and/or titanium dioxide.

In an exemplary embodiment, the invention provides a high protein food bar that may be used to supplement protein intake, particularly in patients undergoing dialysis. The Kidney Disease Outcomes Quality Initiative guidelines recommend a protein intake of ≧1.2 g/kg of body weight/day and established a clinical performance target value for serum albumin of ≧40 g/L or ≧37 g/L (bromcresol green and bromcresol purple laboratory methods, respectively) for dialysis patients. Furthermore, the Centers for Medicare and Medicaid (CMS) expects dialysis providers to intervene as necessary to ensure that more than 81% of patients reach the set albumin target and compliance may impact Medicare reimbursement.

Dietary protein intake (DPI) is reported to be low in patients undergoing hemodialysis, with the mean DPI levels varying from about 0.94 to 1.0 g protein/kg/d.^{3,4,5,6} Hence, the National Kidney Foundation has concluded that approximately half of patients undergoing maintenance dialysis consume less than the recommended amount of protein.⁷ Proposed pay for performance initiatives may impact Medicare reimbursement for dialysis if providers are unable to keep their patients well nourished as measured by serum albumin. Although no single ideal measure of nutritional status exists, the serum albumin concentration is considered to be a useful indicator of protein-energy nutritional status and the extensive literature, in individuals with or without renal failure, relating serum albumin to nutritional status, and the powerful association between hypoalbuminemia and mortality risk amount patients regularly receiving dialysis, strongly support this contention. Albumin is the most abundant plasma protein, maintains osmotic pressure, carries hormones, and serves as an important antioxidant. In addition, the measurement of serum albumin levels is inexpensive, easy to perform, and widely available.

Currently the only renal specific protein sources are liquid or powder, which are known for their less than pleasing flavor. As a result, clinicians have found it difficult to convince their patients to drink these products and thus, compliance has been limited. Additionally, the liquid supplement or powdered supplements, which must be mixed with liquid, add an excess fluid burden, which is detrimental to kidney patients. Unfortunately, many high protein foods are also major sources of phosphorus, hydrogen ions, cholesterol (in the case of animal protein), and dietary fats. In an exemplary embodiment, the present invention provides a tasty (thereby increasing compliance) nutritional supplement that is high in protein and relatively low in sugar (polyols and/or non-nutritive sweeteners may be used to substitute for the sugar), potassium, sodium, phosphorus, calcium and fats.

Therefore, a subject receiving dialysis may be beneficially treated using a multivitamin supplement as described herein and/or a high protein bar as described herein. In an exemplary embodiment, a subject is treated with a composition of the invention throughout the course of their disease progression.

In an exemplary embodiment, the invention provides a high protein source (e.g., a food product or high protein bar) in a ready-to-eat package. Exemplary sources of protein for the high protein bar include, but are not limited to, proteins and amino acids derived from milk, whey, beet; egg, legumes, peanut wheat, soy and combinations thereof. For example, whey protein, soy protein, casein, hydrolyzed beef protein, hydrogenated peanut oil, and combinations thereof. Sweeteners that may be used include, but are not limited to polyols (sugar alcohols), such as maltitol, sorbitol, lactitiol, erythritol, mannitol and xylitol, simple carbohydrates, such as glucose, fructose, galactose, sucrose, lactose and maltose, non-nutritive sweeteners, such as sucralose, aspartame, saccharin, stevioside, tagatose, neotame, and acesulfame potassium. In another exemplary embodiment, the high protein food bar is made using a polyol, with a low sugar content (e.g., about 1 gram per bar), without using any artificial seateners, without vitamin fortification or addition, and having a PDCAAS value of about 1.

In an exemplary embodiment, the high protein bar is formulated so as to contain a minimal quantity of sugar (e.g., less than about 5 g per bar (about 60 grams), less than about 4 g per bar, less than about 3 g per bar, less than about 2 g per bar, or about 1 g of sugar per bar) (polyols and/or non-nutritive sweeteners may be used to substitute for the sugar), potassium, sodium and/or phosphorus. In another exemplary embodiment the high protein bar is formulated to provide a protein source having a biological value higher than that of fish, beef, and/or chicken and the optimal PDCAAS value of about 1. The high protein bar provides an important nutrient source for protein-malnourished, dietary-restricted patients, for example, in dialysis patients/subjects and subjects with chronic states of malnutrition as well. In another exemplary embodiment, the high protein bar is distributed as a Medical food prescribed to the subject by a health care practitioner in order to manage a specific disease of health condition. In another exemplary embodiment, the high protein food bar is made without chicory syrup, cane sugar, caramel, fructose (such as high fructose corn syrup), sucrose, corn syrup, beet sugar and/or any artificial sweetener.

Maintenance dialysis patients are subject to changes in multiple catabolic processes and typically experience a CKD specific form of protein and energy malnutrition, which is characterized by muscle wasting and decreased visceral protein storage. The pathophysiology of muscle wasting in CKD is a complex, multifactorial, process that results in abnormalities in muscle function and exercise performance that begin in earlier stages of CKD and progressively increase through end-stage renal disease. Hence, the high protein food bar of the invention may be used to treat or prevent muscle wasting and/or increase visceral protein storage in CKD patients. In an exemplary embodiment, the high protein food bars are made with about 1 gram of sugar and sweetened using a polyol, since about 50% of such subjects are diabetic, is not fortified with vitamins, and has a PDCAAS score of about 1. Since vitamin regulation in CKD patients is typically based on ingestion of one or more vitamin supplements, the present invention provides a high protein food bar that is not fortified with additional vitamins, therefore the food bar does not unnecessarily add vitamins to the patients existing vitamin supplementation. Artificial sweeteners or non-nutritive sweateners include sucralose, neotame, acesulfame potassium, aspartaine and saccharin.

The Protein Digestibility Corrected Amino Acid Score (PDCAAS) is the currently accepted method for evaluating protein quality. The PDCAAS rankings are determined by comparing the amino acid profile of a protein source against a standard amino acid profile, where the highest possible score is a 1.0. A protein source having a PDCAAS score of I will provide, after digestion of the protein, 100% or more of the indispensable amino acids required by a human. Therefore, the invention provides a high protein food bar where the protein components are blended together to produce a final high protein food bar having a PDCAAS value of about 1. In addition, the high protein food bar may be beneficially made without the addition of added vitamins and minerals. For example, it is beneficial to regulate the vitamin intake of a kidney patient and removal or the lack of additional vitamins in a high energy food bar prevents complication of the vitamin intake as it results from ingestion of the food bar. In contrast to the present invention, common food bars are all prepared with added vitamins, such as vitamin C, which are not helpful to a patient in stage 3, 4, or 5 CKD. By avoiding additional vitamins not naturally present in the ingredients it is possible to produce a high protein food bar that does not have negative impacts on the vitamin regimen of a patient and that supplements or replaces the loss of protein incurred during dialysis.

The high protein food bar of the invention is a specially formulated and processed product intended for the dietary management of a CKD subject that has an impaired capacity digest, absorb, and/or metabolize proteins that provides nutritional support specifically for the management of this need. Optionally, the high protein bar is used under medical supervision and/or is provided to a subject receiving active and/or ongoing medical supervision. For example, the subject may require medical care on a recurring basis, including, receiving instructions on the use of a medical food such as the high protein food bar.

As will now be apparent, the invention provides a suite of kidney products formulated to address the specific needs of kidney patients at precise times during the of their disease progression.

In an exemplary embodiment, the compostions of the invention are supplied to subjects only through the recommendation of a physician and/or dietitian, or by prescription. In another exemplary embodiment the compositions of the invention may be supplied by way of a kidney educational website. In another exemplary embodiment the vitamins are formulated as a capsule for oral administration.

The present invention is further described in the following examples, which are offered by way of illustration.

### EXAMPLE I

Formulation I is a daily multivitamin/multimineral designed specifically to meet the nutritional needs/limitations of subjects with with end stage kidney disease who are on dialysis and comprises a composition containing:

| Compound | Amount | | % RDV |
|---|---|---|---|
| Vitamin C | 120 | mg | 200% |
| Vitamin B1 (Thiamin) | 3 | mg | 200% |
| Vitamin B2 (Riboflavin) | 3.4 | mg | 200% |
| Vitamin B3 (Nicotinic Acid) | 20 | mg | 100% |
| Vitamin B5 (Pantothenic Acid) | 20 | mg | 200% |
| Vitamin B6 (Pyridoxine) | 25 | mg | 1250% |
| Vitamin B12 (Cobalamin) | 2.0 | mg | 33,333% |
| Folic Acid | 5 | mg | 1250% |
| Biotin | 0.3 | mg | 100% |
| NAC | 500 | mg | |
| Zinc | 15 | mg | 100% |
| Chromium | 60 | μg | 50% |
| Selenium | 35 | μg | 50% |
| Vitamin D3 | 400 | IU | 100% |
| Vitamin E | 30 | IU | 100% |

### EXAMPLE II

Formulation II is a daily multivitamin/multimineral designed specifically to meet the nutritional needs/limitations of subjects with stage 3 or stage 4 CKD and comprises a composition containing:

| Compound | Amount | | % RDV |
|---|---|---|---|
| Vitamin C | 120 | mg | 200% |
| Vitamin B1 | 3 | mg | 200% |
| Vitamin B2 | 3.4 | mg | 200% |
| Vitamin B3 | 10 | mg | 50% |
| Vitamin B5 | 10 | mg | 100% |
| Vitamin B6 | 10 | mg | 500% |
| Vitamin B12 | 1.0 | mg | 16,667% |
| Folic Acid | 3 | mg | 750% |
| Biotin | 0.3 | mg | 100% |
| NAC | 500 | mg | |
| Zinc | 15 | mg | 100% |
| Copper | 0.5 | mg | 25% |
| Chromium | 30 | μg | 25% |
| Selenium | 35 | μg | 50% |
| Vitamin D3 | 400 | IU | 100% |
| Vitamin E | 30 | IU | 100% |

### EXAMPLE III

Formulation III is designed to meet the needs of subjects undergoing dialysis by supplying needed protein and comprises a ready-to-eat (i.e., high protein bar) food composition containing:

| | | Grams of protein per 60 g |
|---|---|---|
| Ingredient | Amount (% by wt.) | bar; Total = 20.034 g |
| Coating | 10-30% | 0.276 |
| Water | 5-10% | |
| Glycerine | 10-30% | |
| Maltitol Syrup | 10-30% | |
| Peanut Flour | 5-10% | 1.796 |
| Peanut Extract | 2-5% | |
| Lecithin | less than 2% | |
| Whey Protein Isolate | 10-30% | 6.955 |
| Calcium Caseinate | 10-30% | 5.876 |
| Soy Protein Isolate | 5-10% | 3.927 |
| Wheat Germ | less than 2% | 0.053 |
| Peanuts | 5-10% | 1.124 |
| Almond Butter | less than 2% | 0.027 |
| Salt | less than 2% | |
| Maltodextrin | less than 2% | |
| Tricalcium phosphate | less than 2% | |

This protein bar provides a delicious source of protein that is designed specifically to help meet the protein needs of dialysis patients. Protein malnutrition is one of the most significant problems facing dialysis patients. Dialysis and even pre-dialysis patients often develop anorexia, taste disorders and an aversion to meat, compromising their protein intake and increasing the risk for protein malnutrition.

Each tasty bar provides about 20 grams of highly bioavailable protein with limited simple sugars (sugar alcohols are used in place of sugar, which is especially important for the diabetic patients who are so prevalent in this group), potassium, sodium, calcium, and phosphorus. The high bioavailability of the protein, the high PDCAAS score, and the large amount of protein per unit serving compares very favorably to other rich protein sources such as meat, dairy, and eggs and can provide significantly more protein for the sugar, potassium, calcium, and/or phosphorus load. The protein bars are preferably flavored, for example, chocolate brownie, chocolate peanut butter, peanut butter, lemon flavor, and a raspberry flavor.

This exemplary embodiment provides a peanut butter flavor.

### EXAMPLE IV

Formulation IV is designed to meet the needs of subjects undergoing dialysis by supplying needed protein and comprises a food composition containing:

| | | Grams of protein per 60 g |
|---|---|---|
| Ingredient | Amount (% by wt.) | bar; Total = 20.032 g |
| Coating | 10-30% | 0.281 |
| Water | 5-10% | |
| Glycerine | 10-30% | |
| Maltitol Syrup | 10-30% | |
| Canola Oil | 2-5% | |
| Cocoa | 5-10% | 0.764 |
| Lecithin | less than 2% | |
| Whey Protein Isolate | 10-30% | 7.580 |
| Calcium Caseinate | 10-30% | 5.688 |
| Soy Protein Isolate | 5-10% | 4.554 |
| Soy Crisps | 2-5% | 1.095 |
| Maltodextrin | 2-5% | |
| Wheat Germ | less than 2% | 0.039 |
| Natural Flavor | less than 2% | 0.031 |

In this exemplary embodiment, the protein bar has a Chocolate fudge or chocolate brownie flavor.

### EXAMPLE V

The high protein bars of the invention may be formulated so as to decrease the amount of sugar, sodium, potassium, calcium, and/or phosphate. For example, a high protein bar may have an amount of sodium, potassium, phosphate and/or sugar between about 0.5 mg to about 0.8 mg, about 0.4 mg to about 1.0 mg, or about 0.6 mg to about 0.8 mg per 60 gram bar; calcium between about 50 mg to about 122 mg, about 40 mg to about 200 mg, or about 30 mg to about 300 mg per 60 gram bar; copper between about 0.3 mg to about 0.4 mg, about 0.2 mg to about 0.6 mg, or about 0 mg to about 1 mg per 60 gram bar; magnesium between about 0.2 mg to about 10 mg, about 0.1 mg to about 15 mg, or about 0 mg to about 25 mg per 60 gram bar; phosphate between about 180 mg to about 188 mg, about 150 mg to about 200 mg, or about 100 mg to about 300 mg per 60 gram bar; potassium between about 25 mg to about 113 mg, about 10 mg to about 150 mg, or about 0 mg to about 250 mg per 60 gram bar; sodium between about 213 mg to about 282 ing, about 150 mg to about 350 mg, or about 100 mg to about 400 mg per 60 gram bar; and combinations thereof. For example, a high protein food bar may have less than about 240 mg of sodium and less than about 350 mg of potassium.

The coating of a high protein bar may contain Maltitol powder, palm kernel oil, non fat milk solids, cocoa powder, soya lecithin, salt and natural flavor. Soy Crisps of a high protein bar may contain isolated soy protein, rice flour,n malt extract and salt.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, patents, and patent applications, cited herein are hereby incorporated by reference to the same extent as if each reference were individually and specifically indicated to be incorporated by reference and were set forth in its entirety herein.
¹Farrington K, Miller P, Varghese Z, Baillod R A, Moorhead J F. Vitamin A Toxicity and Hypercalcaemia in Chronic Renal Failure. Br. Med. J. 282:1999-2002, 1981.
²Konstantinos Spargias et al. (2004). Ascorbic Acid Prevents Contrast-Mediated Nephropathy in Patients With Renal Dysfunction Undergoing Coronary Angiography or Intervention. Circulation 110:2837-2842.
³Borah M F, Schoenfeld P Y, Gotch F A, Sargent J A, Wolfsen M, Humphreys M H: Nitrogen balance during intermittent dialysis therapy of uremia. Kidney Int 14:491-500, 1978.
⁴Ikizler T A, Greene J H, Yenicesu M, Schulman G, Wingard R L, Hakim R M: Nitrogen balance in hospitalized chronic hemodialysis patients. Kidney Int Suppl 57:S53-S56, 1996.
⁵Kopple J D, Shinaberger J H, Coburn J W, Sorensen M K, Rubini M E: Optimal dietary protein treatment during chronic hemodialysis. ASAIO Trans 15:302-308, 1969.
⁶Gamba C, Mejia J L, Saldivar S, Pena J C, Correa-Rotter R: Death risk in CAPD patients. The predictive value of the initial clinical and laboratory variables. Nephron 65:23-27, 1993.
⁷National Kidney Foundation Kidney Disease Outcomes Quality Initiative Clinical Practice Guidelines for Nutrition in Chronic Renal Failure. 2000. Available on the world wide web at kidney.org, accessed Jun. 6, 2007.

What is claimed is:

1. A system for the management of renal disease in a subject, the treatment system comprising at least two compositions selected from the group consisting of:
a. a vitamin formulation comprising vitamin C, vitamin D, vitamin E, vitamin B1, vitamin B2, vitamin B3, vitamin B5, vitamin 36, vitamin B12, biotin, vitamin B9, zinc, chromium, and selenium;
b. a composition comprising CoQ10, L-carnitine, lipoic acid, vitamin E, and resveratrol;
c. a composition comprising at least 600 mg of N-acetyl cysteine (NAC) in an enteric coated capsule;
d. a composition comprising phytosterols; and
e. a high protein food bar that has less than about 3 g of sugar per 60 g bar.

2. The treatment system of claim 1, wherein the high protein food bar comprises:
at least about 20 grams of protein per 60 gram high protein food bar;
5-10 % by weight water, 10-30% by weight glycerine, 10-30% by weight manitol syrup, less than about 2% lecithin, 10-30% by weight whey protein, 10-30% by weight calcium caseinate, 5-10% by weight soy protein and less than about 2% wheat germ;
less than about 2 grams of sugar per 60 gram high protein food bar;
less than about 230 mg of sodium; and
less than about 240 mg of potasium.

3. The treatment system of claim 1, wherein the composition comprises about 50 mg of CoQ10 at; L-carnitine in an amount between about 400 mg and about 500 mg; about 50 mg of lipoic acid; about 30 IU of vitamin E; and resveratrol in an amount between about 50 mg and about 100 mg.

4. The treatment system of claim I, wherein the vitamin formulation comprises at least about 500% of the RDV of vitamin B₆, at least about 16,000% of the RDV of vitamin B₁₂, and at least about 1,000% of the RDV of vitamin B₉.

5. The treatment system of claim 1, wherein the composition comprising NAC comprises six enteric coated 600 mg NAC enteric coated capsules and three 500 mg vitamin C capsule in a blister package arranged as a three by three matrix, wherein a first column is marked as being a first dayly dosage, a second column is marked as being a second dayly dosage and a third column is marked as being a third dayly dosage and each column contains three capsules organized as a first NAC enteric coated capsule, a vitamin C capsule and a second NAC enteric coated capsule.

6. A high protein food bar comprising:
at least about 20 grams of protein per 60 grain high protein food bar;
a PDCAAS score of about 1; and
5-10% by weight water, 10-30% by weight glycerine, 10-30% by weight polyol as a sweetener, less than about 2% lecithin, 10-30% by weight whey protein, 10-30% by weight calcium caseinate, 5-10% by weight soy protein and less than about 2% wheat germ.

7. The high protein food bar of claim 6, having less than 2 grains of sugar per 60 gram high protein food bar.

8. The high protein food bar of claim 6, having less than about 230 mg of sodium.

9. The high protein food bar of claim 6, having less than about 240 mg of potassium.

10. The high protein food bar of claim 6, having 1 gram of sugar.

11. The high protein food bar of claim 6, comprising less than about 20% of the Recommended Daily Value of phosphorous.

12. The high protein food bar of claim 6, wherein the food bar is contained in a wrapper label that states the food bar is intended to be used to manage protein needs for a patient undergoing dialysis or a patient suffering from diabetes.

13. The high protein food bar of claim 6, wherein the food bar comprises an outer layer made with maltitol powder, palm kernel oil, non fat milk solids, cocoa powder, soya lecithin, salt and natural flavor.

14. The high protein food bar of claim 6, wherein no chicory syrup, cane sugar, caramel, fructose, sucrose, corn syrup, or beet sugar is added to the high protein food bar.

15. A method of treating skeletal muscle wasting in a subject, the method comprising:
providing a subject with a protein source formulated for the management of the nutrient needs that result from kidney disease;
administering the protein source comprising at least about 20 grams of protein per 60 gram high protein food bar having a PDCAAS score of about 1, about 1 gram of sugar, less than about 230 mg of sodium and less than about 240 mg of potassium; wherein the high protein food bar is sweetened with a polyol and is free of added vitamins to the subject, thereby compensating for protein depleation in the subject.

16. The method according to claim 15, wherein the high protein food bar does not contain a substance regulated by the United States Food and Drug Administration.

17. The method according to claim 15, wherein the high protein food bar is sweetened with a polyol.

18. The method according to claim 17, wherein the polyol is maltitol.

19. A composition comprising Vitamin E, Alpha lipoic acid, L-Carnitine, Coenzyme Q10, and Resveratrol.

20. The composition of claim 19, wherein the composition is contained in a capsule.

21. The composition of claim 20, comprising 30 internation units of Vitamin E, about 50 mg of alpha lipoic acid, about 400 mg of L-Carnitine, about 50 mg of Coenzyme Q10, and about 50 mg of resveratrol.

22. A method for treating or preventing weakness, fatigue or cramping associated with kidney disease, the method comprising administering a composition comprising Vitamin E, Alpha lipoic acid, L-Carnitine, Coenzyme Q10, and Resveratrol, to a subject suffering from weakness, fatique or cramping associated with kidney disease.

23. A vitamin formulation comprising:
a capsule comprising vitamin C, vitamin D₃, vitamin E, vitamin B₁, vitamin B₂, vitamin B₃, vitamin B₅, vitamin B₆, vitamin B₁₂, biotin, vitamin B₉, copper, zinc, chromium, and selenium, wherein the vitamin B₁₂ is present in an amount of at least 16,667% ofthe RDV per capsule.

24. The vitamin formulation of claim 23, wherein the vitamin B₉ is present in an amount of at least about 750% of the RDV.

25. The vitamin formulation of claim 24, wherein composition provides about 200% of the Recommended Daily Value (RDV) of vitamin B₅, about 1,250% of the RDV of vitamin B₆, about 33,333% of the RDV of vitamin B₁₂, and about 1,250% of the RDV of vitamin B₉.

26. The vitamin formulation of claim 24, wherein the composition comprises 100% of the Recommended Daily Value (RDV) of vitamin B₅, about 500% of the RDV of vitamin 36, about 16,667% of the RDV of vitamin B₁₂, and about 750% of the RDV of vitamin B₉.

27. A method of managing a subject diagnosed with chronic renal disease, comprising administering a capsule comprising vitamin C, vitamin D₃, vitamin E, vitamin B₁, vitamin B₂, vitamin B₃, vitamin B₅, vitamin B₆, vitamin B₁₂, biotin, vitamin B₉, copper, zinc, chromium, and selenium, wherein the vitamin B₁₂ is present in an amount of at least 16,667% of the RDV per capsule and wherein the formulation of the composition changes at least once as the subject's disease state changes.

28. The method according to claim 27, further comprising administering a second composition comprising CoQ10, L-carnitine, lipoic acid, vitamin E and resveratrol, to a subject suffering from weakness, fatique or cramping associated with kidney disease.

29. The method according to claim 28, wherein the composition comprises administering an enteric coated capsule providing about 200% of the Recommended Daily Value (RDV) of vitamin B₅, about 1,250% of the RDV of vitamin B₆, at least about 33,000% of the RDV of vitamin B₁₂, and about 1,250% of the RoV of vitamin B₉.

30. The method according to claim 27, wherein the composition comprises administering an enteric coated capsule providing 100% of the Recommended Daily Value (RDV) of vitamin B₅, about 500% of the RDV of vitamin B₆, at least about 16,000% of the RDV of vitamin B₁₂, and about 750% of the RDV of vitamin B₉.
